(19) United States
(12) Patent Application Publication
(10) Pub. No.: US 2011/0263701 A1
(43) Pub. Date: Oct. 27, 2011

(54) GABAPENTIN ENACARBIL COMPOSITIONS

(76) Inventors: Sigal BLAU, Raanana (IL); Ilan ZALIT, Rosh Ha Ayin (IL)

(21) Appl. No.: 13/090,823

(22) Filed: Apr. 20, 2011

Related U.S. Application Data
(60) Provisional application No. 61/326,447, filed on Apr. 21, 2010.

(51) Int. Cl. A61K 31/27; A61P 25/08

(52) U.S. Cl. 514/484

(57) ABSTRACT

The present invention provides a stabilized composition comprising a non-crystalline gabapentin enacarbil and at least one crystallization-inhibiting compound. In particular, the present invention provides a stabilized composition of gabapentin enacarbil, wherein the gabapentin enacarbil is maintained in a non-crystalline form by the composition, for example, as an amorphous form. The invention also provides, among other things, methods of making the stabilized composition, or use of the stabilized composition for making a medicament.

This patent application claims the benefit of U.S. Provisional Patent Application No. 61/326,447 filed Apr. 21, 2010, the disclosure of which is hereby incorporated by reference.

### BACKGROUND OF THE INVENTION

Gabapentin enacarbil (GABA-E), 1-{[(alpha-isobutanoyloxyethoxy)carbonyl]-aminomethyl}-1-cyclohexane acetic acid, CAS number 478296-72-9, is a prodrug of the widely used anticonvulsant and analgesic agent gabapentin.

Apparently, gabapentin enacarbil was developed in order to overcome an unfavorable pharmacokinetic profile of gabapentin. U.S. Pat. No. 6,818,787 describes a prodrug of gabapentin, which allegedly is absorbed throughout the entire length of the intestine and exhibits a dose proportional pharmacokinetics.

US2005/0154057 describes a crystalline form of 1-{[(alpha-isobutanoyloxyethoxy)carbonyl]aminomethyl}-1-cyclohexane acetic acid. According to US2005/0154057, crystalline forms of drugs are, in general, preferred over the amorphous forms, in part, because of their superior stability, e.g. in many situations, an amorphous drug converts to a crystalline form upon storage. They further mention that amorphous solids and particularly hygroscopic solids are difficult to handle under pharmaceutical processing conditions because of low bulk densities and unsatisfactory flow properties.

There is a need in the art to provide further formulations of gabapentin enacarbil. In the present invention, the inventors disclose a stabilized pharmaceutical composition comprising a non-crystalline gabapentin enacarbil (GABA-E).

### SUMMARY OF THE INVENTION

In a first aspect, the present invention provides stabilized composition comprising a non-crystalline gabapentin enacarbil and at least one crystallization-inhibiting compound. In particular, the present invention provides a stabilized composition of gabapentin enacarbil, wherein the gabapentin enacarbil is maintained in a non-crystalline form by the composition, for example, as an amorphous form.

According to a second aspect, the invention provides a stabilized composition comprising a non-crystalline form of gabapentin enacarbil and at least one crystallization-inhibiting compound.

In a preferred embodiment, the invention provides a composition comprising non-crystalline gabapentin enacarbil, preferably a stabilized composition, and at least one crystallization-inhibiting compound, characterized in that, on solids basis, the gabapentin enacarbil/crystallization-inhibiting compound weight ratio is about 1 or more, preferably about 1.5 or more, more preferably about 2 or more, most preferably about 4 or more, and even most preferably about 6 or more.

According to a third aspect, the invention provides a method for manufacturing a stabilized composition comprising non-crystalline gabapentin enacarbil and at least one crystallization-inhibiting compound. In particular, the process comprises: the steps of: (1) providing a solution comprising gabapentin enacarbil and the at least one crystallization-inhibiting compound in at least one organic solvent; and (2) removing the solvent(s). For example, the method may comprise the steps of dissolving the gabapentin enacarbil in a solvent, mixing or dissolving at least one crystallization-inhibiting compound in a solvent, mixing the two mixtures and removing the solvents, for example by evaporation, spray drying, freeze drying or similar process.

The compositions may also be prepared by a process comprising: (1) providing a melted mixture of the crystallization-inhibiting compound and gabapentin enacarbil; and (2) solidifying the melted mixture by cooling.

Also provided are pharmaceutical formulations and dosage forms comprising the compositions as well as processes for the preparation of the pharmaceutical formulations and dosage forms.

The present invention aims to overcome the difficulties of formulating a non-crystalline active agent, namely gabapentin enacarbil.

### BRIEF DESCRIPTION OF THE DRAWINGS

FIG. 1: Powder X-ray diffraction pattern of crystalline gabapentin enacarbil (GABA-E).

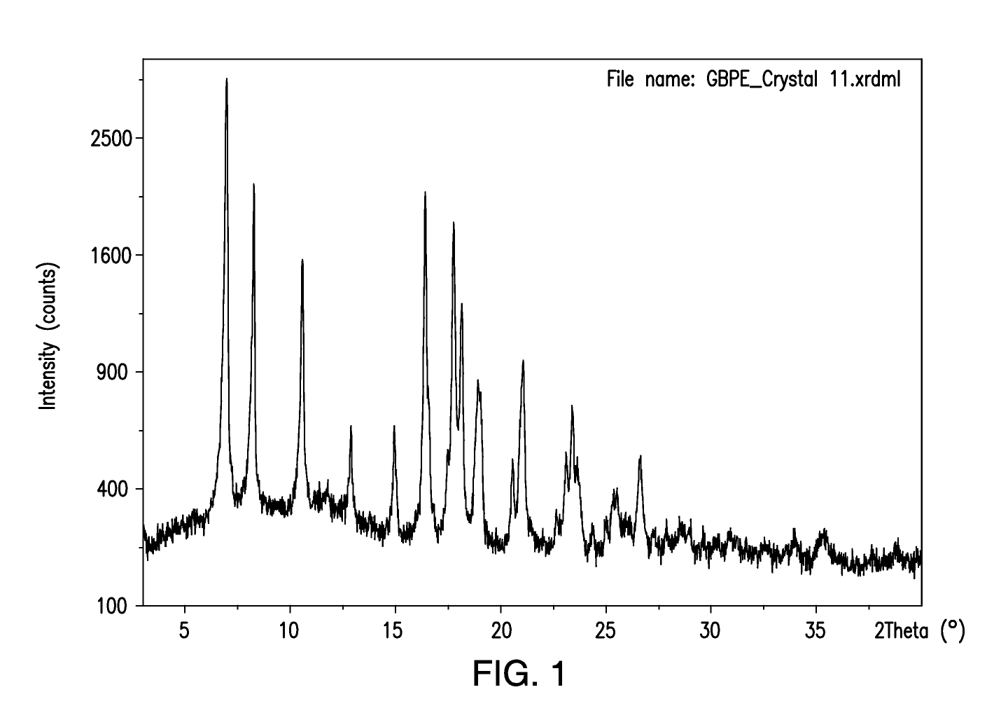

FIG. 2:

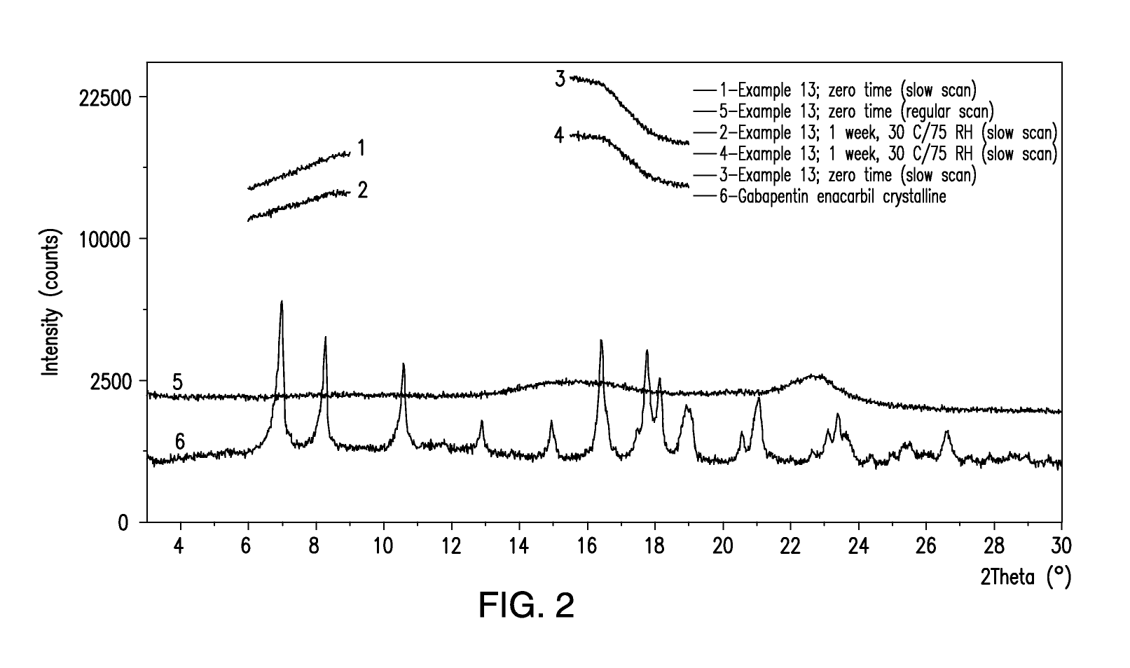

Powder X-ray diffractogram of tablets prepared according to Example 13: (1, 3) (slow scan) and (5) (regular scan) before storage at 30° C. and 75% relative humidity (“RH”) for 1 week (=zero time); (2, 4) (slow scan) after storage at 30° C. and 75% RH for 1 week; and (6) powder X-ray diffractogram of crystalline gabapentin enacarbil (the slow scans are fragmented because the measurements were done on the range where GABA-E has highest peak and where first peak of crystalline GABA-E appeared when crystallisation start).

FIG. 3: Powder X-ray diffractogram of tablets prepared according to Example 14, after storage at:

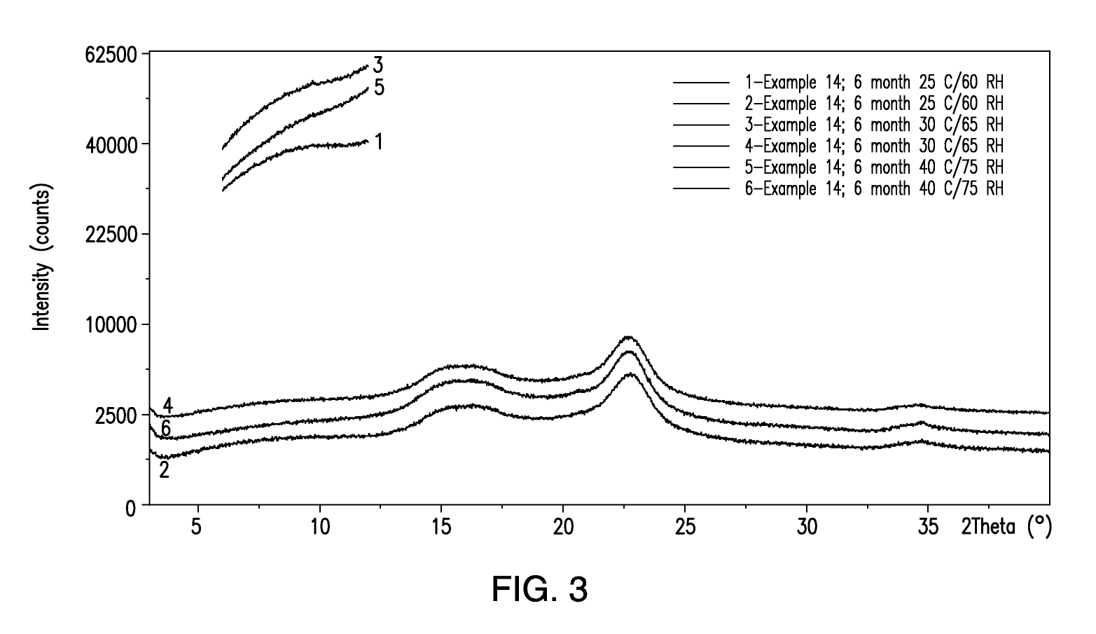

(1)(slow scan) and (2)(regular scan): 25° C./60% RH for 6 months

(3)(slow scan) and (4)(regular scan): 30° C./65% RH for 6 months

(5)(slow scan) and (6)(regular scan): 40° C./75% RH for 6 months

(the slow scans were fragmented because the measurements were done on the range where GABA-E had the highest peak and where the first peak of crystalline GABA-E appeared when crystallization started)

FIG. 4: XRD of tablets prepared according to Example 15, after storage at:

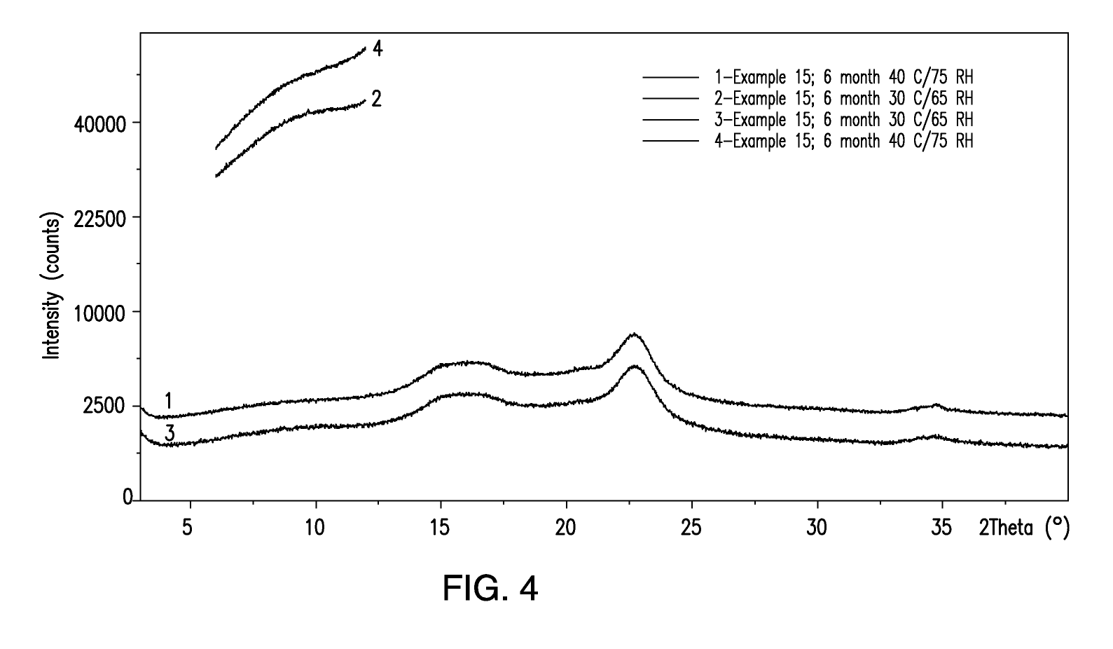

(1) (regular scan) and (4) (slow scan): 40° C./75% RH for 6 months

(2) (slow scan) and (3) (regular scan) 30° C./65% RH for 6 months

(the slow scans are fragmented because the measurements were done on the range where GABA-E had the highest peak and where the first peak of crystalline GABA-E appeared when crystallization started)

### DETAILED DESCRIPTION OF THE INVENTION

When the drug is unstable because of crystallization, for low drug load pharmaceutical compositions, it might be possible to use a high concentration of a crystallization-inhibiting compound and still provide a formulation suitable for oral administration, but when a pharmaceutical composition is required to contain high dose of active ingredient, e.g. more than 300 mg, it is necessary to provide a formulation with a minimum amount of an acceptable pharmaceutical ingredients. The present invention relates to compositions comprising a non-crystalline gabapentin enacarbil. In particular, the present invention enables the gabapentin enacarbil to be stabilized in a solid, non-crystalline form, even at a high ratio of gabapentin enacarbil, and at least one crystallization-inhibiting compound, e.g. about 1 or more, preferably about 1.5 or more, more preferably about 2 or more, most preferably about 4 or more, and even most preferably about 6 or more, to 1 based on the amount by weight of the gabapentin enacarbil to the at least one crystallization-inhibiting compound.

All weight ratios in the present invention of gabapentin enacarbil to crystallization-inhibiting compound are given free of solvents, i.e. “dry” weights.

The present invention relates to a non-crystalline form of gabapentin enacarbil, preferably wherein the non-crystalline form of gabapentin enacarbil is substantially free of any crystalline forms. By “substantially free” is meant 20% or less by weight, for example about 10% or less, about 5% or less, about 2% or less, about 1% or less, or between about 1% and about 20%, between about 2% and about 10%, between about 1% and about 2%.

“Non-crystalline” and “not in crystalline form” mean materials that do not produce X-ray powder diffraction patterns having peaks characteristic of crystalline gabapentin enacarbil, when performed on X-ray powder diffraction (XRPD) with Philips X'Pert PRO powder diffractometer equipped with X'Celerator detector, X-ray tube PW3373/00; Cu anode LFF, CuKα radiation, λ=1.54184 Å at 295±5K. Prior to analysis the samples were applied directly on silicon PW 1817/32 “zero background” holder. The scanning parameters were: range: 0-30 degrees two-theta; scan mode: continuous scan; step size (regular scan): 0.0167; and scan speed (regular scan): 0.05°/sec, step size (slow scan): 0.0167; and scan speed (slow scan): 0.002°/sec. Amorphous gabapentin enacarbil is an example of non-crystalline gabapentin enacarbil.

In a first aspect, the present invention provides a stabilized composition of gabapentin enacarbil, wherein the gabapentin enacarbil is in a non-crystalline form, preferably as an amorphous form. In particular, the stabilized composition comprises a non-crystalline gabapentin enacarbil and at least one pharmaceutically acceptable crystallization-inhibiting compound.

Preferably, the composition comprising a non-crystalline gabapentin enacarbil and at least one crystallization-inhibiting compound, is characterized in that the non-crystalline gabapentin enacarbil to crystallization-inhibiting compound weight ratio is at least about 1, preferably about 1.5 or more, more preferably about 2 or more, most preferably about 4 or more, and most preferably about 6 or more, to 1. In other preferred embodiments, the weight ratio of the gabapentin enacarbil to the crystallization-inhibiting compound is about 1:1 to about 8:1, about 1:1 to about 6:1, about 1.5:1 to about 10:1, about 1.5: to about 8:1 about 1.5:1 to about 6:1, about 2:1 to about 10:1, about 2:1 to about 8:1; or about 2:1 to about 6:1. For example, between about 1:1 and about 10:1, between about 1:1 and about 5.5:1, between about 1:1 to about 2:1 and between about 1:1 to about 1.5:1 of gabapentin enacarbil to crystallization-inhibiting compound.

As used herein, a numerical range “between” a lower number and a higher number means that the numerical range includes the lower number as the lower end and the higher number as the higher end. For instance, a weight ratio “between about 1:1 and about 10:1” means that the weight ratio is about 1:1, about 10:1 or any number less larger than about 1:1 and smaller than about 10:1.

In another preferred embodiment, the composition comprises a non-crystalline gabapentin enacarbil in an amount of between about 50% to about 95% by weight of the composition (of total amount of gabapentin enacarbil and crystallization-inhibiting compound), e.g. about 60% to about 95%, about 65% to about 95%, about 70% to about 95%, about 80% to about 90% by weight of the composition. The remainder may be the at least one crystallization-inhibiting compound. Preferably, the gabapentin enacarbil is present in an amount of about 55% to about 85%, more preferably about 60% to about 80% by weight of the composition comprising a non-crystalline gabapentin enacarbil and at least one crystallization-inhibiting compound.

In another preferred embodiment, the composition consisting essentially of a non-crystalline gabapentin enacarbil and at least one crystallization-inhibiting compound.

By the term “stable” or “stabilized” it is meant that the non-crystalline form of gabapentin enacarbil does not convert into crystalline forms. For example at manufacture (Time Zero), after 1 week storage at 25° C. or 30° C. and 75% relative humidity, and/or after 1 month storage at 25° C. and 60% relative humidity, the compositions of the present invention comprise not more than the lower detection limit of crystalline gabapentin enacarbil, e.g. not more than 1% by weight as measured using x-ray powder diffraction according to the method described above. Preferably the term “stable” or “stabilized” means the gabapentin enacarbil does not convert into crystalline form:
(i) at manufacture (time zero), and/or after 3 months of storage at 25° C. and 60% relative humidity (RH), or preferably
(ii) at manufacture (time zero), and/or after 6 months of storage at 25° C. and 60% relative humidity (RH), or preferably
(iii) at manufacture (time zero), and/or after 3 months of storage at 30° C. and 65% relative humidity (RH), or preferably
(iv) at manufacture (time zero), and/or after 6 months of storage at 30° C. and 65% relative humidity (RH), or preferably
(v) at manufacture (time zero), and/or after 3 months of storage at 40° C. and 75% relative humidity (RH), or preferably
(vi) at manufacture (time zero), and/or after 6 months of storage at 40° C. and 75% relative humidity (RH), or preferably
(vii) at manufacture (time zero), and/or after 12 months of storage at 40° C. and 75% relative humidity (RH).

In any embodiment of the present invention, the at least one crystallization-inhibiting compound may comprise between 1 and 5 crystallization-inhibiting compounds, preferably between 1 and 3, more preferably 1 or 2 crystallization-inhibiting compounds, most preferably 1 crystallization-inhibiting compound.

Preferably, the crystallization-inhibiting compound (“crystallization inhibitor” or “crystallization inhibitor substance”) refers to a substance which can be used to prepare a stabilized non-crystalline gabapentin enacarbil composition. For example, the crystallization-inhibiting compound particularly refers to a substance which when formed as an intimate admixture/solid solution with the non-crystalline gabapentin enacarbil, stabilizes the non-crystalline form of gabapentin enacarbil. In other words, the crystallization inhibiting compound or substance is able to substantially prevent the formation of crystalline gabapentin enacarbil, and preferably maintains the gabapentin enacarbil in a non-crystalline form during storage (e.g. over the time periods and storage conditions as indicated above).

In particular, the present invention is based on the surprising finding that certain pharmaceutically acceptable excipients, such as pharmaceutically acceptable polymers, gums, oils, waxes, fatty acids, and fatty acid esters, are able to stabilize non-crystalline gabapentin enacarbil. In particular, these excipients are especially effective in stabilizing non-crystalline gabapentin enacarbil when they are used to prepare intimate admixtures/solid solutions with non-crystalline gabapentin enacarbil, Thus, these excipients enable the production of stabilized non-crystalline gabapentin enacarbil compositions. In the stabilized composition according to the first aspect of the invention, the non-crystalline gabapentin enacarbil is typically stabilized by being in an intimate admixture with the at least one crystallization inhibitor.

Thus, in any embodiment of the present invention, there is provided a stabilized composition comprising a non-crystalline gabapentin enacarbil and at least one crystallization-inhibiting compound. Preferably, the present invention further provides a composition comprising a homogeneous mixture (preferably an intimate admixture or solid solution) of non-crystalline gabapentin enacarbil and at least one crystallization-inhibiting compound. More preferably, the present invention further provides a composition comprising a homogeneous mixture (preferably an intimate admixture or solid solution) of non-crystalline gabapentin enacarbil and at least one pharmaceutically acceptable excipient selected from the group consisting of a polymer, including hydrophobic and hydrophilic polymers [for example, water-soluble polymers [such as pyrrolidone polymers, e.g. polyvinylpyrrolidone (povidone, e.g. Povidone K25, K30, K60, K90 and K120, VA64, particularly Povidone K30 and VA64), water-insoluble polymers (such as cross-linked polyvinylpyrrolidone, i.e. crospovidone), and cellulose-based polymers, [such as alkyl cellulose ethers—particularly ethylcellulose (especially ethylcellulose having various viscosities, such as Ethocel 7 cP, Ethocel 10 cP, Ethocel 20 cP, Ethocel 50 cP, Ethocel 100 cP, especially Ethocel 7 cP and Ethocel 100 cP), hydroxyethylcellulose, hydroxypropylcellulose (e.g. Klucel LF, Klucel MF, Klucel GF, Klucel JF, Klucel LF and Klucel EF, particularly Klucel LF), and hydroxypropylmethyl cellulose, such as Pharmacoat 606)], waxes [especially beeswax, carnauba wax, and microcrystalline wax]; gums (such as xanthan gum, guar gum, agar, carrageenan, tragacanth or acacia), oils (e.g. hydrogenated castor oil), fatty acids, and fatty acid esters (for example glycerol fatty acid esters, including mono-, di- and tri-glycerides, particularly glyceryl monostearate—particularly glyceryl behenate and glyceryl monostearate 402BP and glycerylmonostearate 40-55BP).

In any embodiment of the present invention the crystallization inhibiting compound is a pharmaceutically acceptable excipient that is a crystallization inhibiting compound, i.e. the compositions of the present invention comprise a solid, non-crystalline gabapentin enacarbil (e.g. amorphous gabapentin enacarbil) and at least one pharmaceutically acceptable crystallization-inhibiting compound. The stabilized compositions according to any embodiment of the present invention are suitable for the preparation of pharmaceutical dosage forms.

In any embodiment of the present invention, the crystallization-inhibiting compound is a pharmaceutically acceptable crystallization-inhibiting compound. Preferably, in any embodiment of the present invention, the crystallization-inhibiting compound is selected from the group consisting of polymers (including hydrophobic and hydrophilic polymers, and particularly a cellulose-based polymer), waxes, gums, oils, fatty acids, and fatty acid esters. The crystallization-inhibiting compound preferably includes but is not limited to hydrophilic or water soluble polymers, water-insoluble polymers, oils and mixtures thereof. For example hydrophilic or water soluble polymers include celluloses such as methylcellulose, carboxymethyl cellulose, hydroxypropyl methylcellulose (e.g. Pharmacoat 606), Hydroxypropyl Cellulose, cross-linked sodium carboxymethyl cellulose and cross-linked hydroxypropyl cellulose; polyvinylpyrrolidone; copovidone; cross-linked polyvinylpyrrolidone; polyvinyl acetate; polyvinyl alcohol; gums including natural gums, for example, those selected from the group consisting of: gum arabic, gum ghatti, gum karaya, gum tragacanth; hydrophilic colloids such as alginates (e.g. sodium, potassium, magnesium, or ammonium alginates); and a combination thereof. For example water-insoluble polymers include celluloses such as ethylcellulose, cellulose acetates, and their derivatives, cellulose acetate phthalate, hydroxyl propyl methylcellulose phthalate, cellulose acetate, cellulose diacetate, polymers, polymethacrylic acid based polymers and cationic copolymers of ethylacrylate and methylacrylates with quarternary ammonium groups sold as Eudragit RL and RS, Ethylacrylate methylmethacrylate copolymer with neutral ester groups sold as NE-30D, copolymers of the above polymers; and a combination thereof. For example, oils include hydrogenated castor oil; waxes such as beeswax, carnauba wax, and microcrystalline wax; fatty alcohols such as, stearyl alcohol, cetyl alcohol, and myristyl alcohol; fatty acid esters such as glyceryl monostearate, glycerol distearate, glycerol monooleate, acetylated monoglycerides, tristearin, tripalmitin, cetyl esters wax, glyceryl palmitostearate, and glyceryl behenate; and a combination thereof.

Suitable crystallisation inhibitors for the compositions of any embodiment of the present invention are preferably selected from the group consisting of:
water soluble polymers (preferably polyvinylpyrrolidone, including the particular grades of povidone as specified above);
water insoluble polymers (preferably cross-linked polyvinylpyrrolidone, including the particular crospovidone grades as specified above);
oils (especially hydrogenated castor oil);
cellulose-based polymers, especially alkyl cellulose ethers (particularly ethylcellulose, hydroxyethylcellulose, hydroxypropylcellulose and hydroxypropylmethyl cellulose, including the particular grades of these polymers as specified above);
fatty acid esters, especially glycerol fatty acid esters (especially mono-, di- and tri-glycerides, particularly glyceryl behenate and glyceryl monostearate, including those specified above); and
mixtures thereof.

Particularly, the at least one crystallization-inhibiting compound includes glyceryl monostearate (especially glyceryl monostearate 402BP and glyceryl monostearate (40-55BP), hydrogenated castor oil, polyvinylpyrrolidone (especially Povidone K30, Povidone K90, Povidone VA64), cross-linked polyvinylpyrrolidone (especially copovidone), celluloses (cellulose ethers) such as ethylcellulose (especially Ethocel 7 cP, Ethocel 100 cP), hydroxypropyl cellulose (especially Klucel LF), hydroxypropyl methylcellulose or combinations thereof.

Preferably, the at least one crystallization-inhibiting compound includes glyceryl monostearate (especially glyceryl monostearate 402BP and glyceryl monostearate (40-55BP), hydrogenated castor oil, polyvinylpyrrolidone (especially Povidone K30, Povidone K90, Povidone VA64), celluloses such as ethylcellulose (especially Ethocel 7 cP, Ethocel 100 cP), hydroxyl propyl methylcellulose or combinations thereof. More preferably, the at least one crystallization-inhibiting compound includes polyvinylpyrrolidone, celluloses such as ethylcellulose and hydroxyl propyl methylcellulose, or combinations thereof. Particularly preferred are crystallizing inhibitors selected from the group consisting of ethyl cellulose, glycerylmonostearate, hydrogenated castor oil, hydroxypropyl cellulose, glyceryl behenate polyvinylpyrrolidone and hydroxypropyl methyl cellulose (e.g. Pharmacoat 606), and especially polyvinyl pyrrolidone and ethyl cellulose, and more preferably polyvinyl pyrrolidone.

In any embodiment of the present invention, the composition consists essentially of a non-crystalline gabapentin enacarbil and at least one crystallization-inhibiting compound as described above.

In some cases, the above compositions can advantageously include a surfactant. Thus, in any embodiment of the present invention, the composition may further comprise or consist essentially of at least one surfactant.

Suitable surfactants include, but are not limited to: non ionic surfactants such as: polyoxyethylene sorbitan fatty acid esters, fatty acid esters of alcohols and copolymers of ethylene oxide and propylene oxide; anionic surfactants such as: sodium lauryl sulfate and docusate sodium; and amphoteric agents such as phospholipids and polypeptides. Sodium lauryl sulfate is a particularly preferred surfactant.

In a preferred embodiment, said gabapentin enacarbil is in intimate admixture with at least one crystallization-inhibiting compound. As used herein “intimate admixture” is a mixture of closely packed components as opposed to simple powder blends. Thus, the term “intimate admixture” typically refers to a mixture wherein the non-crystalline gabapentin enacarbil and the one or more crystallisation inhibitors are homogeneously mixed on a molecular level, as opposed to a mixture obtained by physical mixing of the two components in solid form.

Two examples of such intimate admixtures include a co-precipitate of gabapentin enacarbil and the at least one crystallization-inhibiting compound from a solvent and gabapentin enacarbil in solid solution with at least one crystallization-inhibiting compound. Preferably, the non-crystalline gabapentin enacarbil is prepared in situ in the composition. Thus, preferably the non-crystalline gabapentin enacarbil is prepared in the presence of the at least one crystallisation inhibitor. For example a mixture (e.g. solution) comprising gabapentin enacarbil and the one or more crystallisation inhibitors can be co-evaporated/co-precipitated, or a solid mixture of gabapentin enacarbil and at least one crystallisation inhibitor can be co-melted.

The stabilized compositions of non-crystalline gabapentin enacarbil according to any of the embodiments described herein may be used to prepare pharmaceutical compositions such as pharmaceutical dosage forms. Thus, the invention further provides the use of the stabilized compositions according to any of the embodiments described herein in the manufacture of a pharmaceutical dosage form.

In a further aspect, the present invention describes a stabilized pharmaceutical compositions of non-crystalline gabapentin enacarbil according to all described above. Preferred pharmaceutical compositions according to the present invention are compressed dosage forms, e.g. tablets. Thus, a further aspect of the present invention, provides a pharmaceutical dosage form (e.g. in the form of a tablet or capsule, preferably a tablet) comprising a stabilized composition of non-crystalline gabapentin enacarbil according to any embodiment of the present invention as described herein.

In a preferred embodiment the pharmaceutical compositions comprises a non-crystalline gabapentin enacarbil, at least one crystallization-inhibiting compound, at least one carrier and at least one lubricant. As described herein, the non-crystalline gabapentin enacarbil and the at least one crystallization-inhibiting compound is in the form of an intimate admixture (as discussed herein), which may be admixed or blended with the carrier(s) and lubricant(s).

Thus, preferred dosage forms according to the present invention comprise a pharmaceutical composition of non-crystalline enacarbil as described in any embodiment of the present invention, and at least one carrier and at least one lubricant.

Suitable carriers includes but not limited to microcrystalline cellulose (e.g. Avicel PH105®, Avicel® PH102, Avicel® PH101, and Avicel® PH113), lactose, mannitol, sorbitol, xylitol, sucrose, talc, dicalcium phosphate and starch. Particularly preferred as carriers are microcrystalline cellulose (such as e.g. Avicel PH105®, Avicel® PH102, Avicel® PH101, and Avicel® PH113),

Suitable lubricants and glidants includes but not limited to magnesium stearate, stearic acid, silicon dioxide, aluminum, zinc and calcium stearates, hydrogenated vegetable oils, talc and silicones. Preferably, the lubricant is selected from the group consisting of magnesium stearate, stearic acid, silicon dioxide, aluminum, zinc and calcium stearates, hydrogenated vegetable oils, and talc. More preferably, the lubricant and glidants are selected from the group consisting of silicon dioxide, magnesium stearate and talc. Particularly preferred lubricants and glidants are silicon dioxide and magnesium stearate.

In a preferred embodiment the pharmaceutical compositions comprise a non-crystalline gabapentin enacarbil, at least one crystallization-inhibiting compound and at least one release retarding ingredient, wherein the composition releases the gabapentin enacarbil over a predetermined time period. Thus, the present invention further provides a dosage form comprising the composition containing non-crystalline gabapentin enacarbil and a crystallization inhibiting compound according to any embodiment described herein, and at least one release-retarding ingredient. Such dosage forms of the present invention provide a controlled-release of the non-crystalline gabapentin enacarbil, i.e. release of the active agent over a predetermined time period.

Suitable release-retarding ingredients include, but not limited to, hydrophilic or water soluble polymers, water-insoluble polymers, oils, waxes, fatty acid esters, and mixtures of any two or more thereof. For example, suitable release-retarding ingredients include, but not limited to, hydrophilic or water soluble polymers, water-insoluble polymers, oils and mixtures of any two or more thereof. For example, suitable release-retarding ingredients include various natural gums (e.g. xanthan gum, guar gum, agar, carrageenan, tragacanth or acacia), waxes (such as carnauba wax, hydrogenate vegetable oils such as hydrogenated castor oil) , hydrophilic polymers (e.g. cellulose derivatives such as carboxymethylcellulose, hydroxypropyl cellulose or hydroxypropylmethyl cellulose), other cellulose ethers such as ethyl cellulose, cellulose acetate, cellulose diacetate, cellulose triacetate, polyvinyl alcohol, polyvinylpyrrolidone, copovidone, polyethylene oxide, alginic acid or salts thereof, polyvinyl acetate, glyceryl esters of fatty acids (e.g. glyceryl behenate), polymethacrylates, and polyethylene glycol. Preferably, the release retarding ingredient is a cellulose derivative, such as a cellulose ester, more preferably hydroxypropylmethyl cellulose (such as hypromellose having a viscosity of 80,000 to 120,000 mPas, e.g. Methocel® K100M), glyceryl behenate, and copovidone.

In a further aspect, the present invention provides a method for manufacturing a composition comprising non-crystalline gabapentin enacarbil and at least one crystallization-inhibiting compound. The procedure described below is suitable for preparing the composition as defined in any embodiment disclosed herein. In this aspect, the method generally involves forming a solution comprising gabapentin enacarbil and the at least one crystallization-inhibiting compound, and coprecipitation of the components by removing the solvent, e.g. by evaporation (including evaporation at atmospheric or reduced pressure and flash evaporation), spray drying, and freeze drying. Preferably, the removal of the solvent is by evaporation/flash evaporation (either at atmospheric or reduced pressure), or by freeze drying. Preferably, the method comprises steps of dissolving the gabapentin enacarbil in at least one first solvent (preferably ethanol, or acetone, more preferably acetone), mixing or dissolving at least one crystallization-inhibiting compound in at least one second solvent (preferably ethanol or acetone), mixing the two mixtures and removing the solvents, for example by evaporation, spray drying, freeze drying or similar process. Preferably, the at least one crystallization-inhibiting compound is dissolved.

Preferably, the solvents are removed by evaporation (i.e. by heating). The evaporation may be at atmospheric pressure or reduced pressure (i.e. a pressure of less than 1 atmosphere).

The evaporation temperature depends on the solvent(s) and the pressure. Typically, the evaporation temperature is between about 5 to about 130° C., preferably about 10° C. to about 130° C., about 25° C. to about 120° C., about 30° C. to about 100° C., about 40° C. to about 90° C., about 45° C. to about 80° C., about 45° C. to about 70° C., about 45° C. to about 65° C. or about 45° C. to about 55° C.

The evaporated mixture may optionally be milled. Optionally, the method includes milling of the dry mixture, wherein the milling is preferably performed below 10° C., more preferably 8° C. or below, 5° C. or below, and particularly 4° C. or below)—typically the milling is carried out at a temperature between about −10° C. to about 8° C., or −5° C. to about 8° C., or 0° C. to about 5° C.

Preferably, the milling step is carried out after the evaporated mixture is cooled to below 10° C. (more preferably 5° C. or below, and particularly 4° C. or below).—typically between about −10° C. to about 8° C., or −5° C. to about 8° C., or 0° C. to about 5° C. Preferably the cooling is carried out at these temperatures for a period of between about 10 minutes to about 24 hours, more preferably about 30 minutes to about 3 hours, or 1-2 hours.

According to a preferred embodiment, the method further comprises steps for manufacturing a pharmaceutical composition, including compressing the mixture to form a tablet. For example, the method for manufacturing a pharmaceutical composition may include combining the stabilized composition of non-crystalline gabapentin enacarbil and at least one crystallization inhibitor as described in any of the embodiments disclosed herein, with at least one pharmaceutically acceptable excipient (preferably at least one filler) and with a lubricant (preferably silica). The composition can then be compressed into tablets, or minitablets for filling into capsules.

For the preparation of the stabilized compositions of non-crystalline gabapentin enacarbil and at least one crystallization inhibitor as described in any embodiment or aspect of the present invention, the at least one first solvent may be any suitable solvent which is able to dissolve the gabapentin enacarbil. A second solvent used with the active ingredient may be similar (e.g. the same) or different from the first solvent used with the crystallization inhibiting compound with the proviso that it does not precipitate the active ingredient. Thus, preferably when a single solvent is used, both the gabapentin enacarbil and the at least one crystallisation inhibitor compound are soluble in the solvent, and when two or more solvents are used, the solvents are able to maintain both the gabapentin enacarbil and the crystallization inhibiting compound in solution. A surfactant as described below may be added to the mixture, or to the solution of the crystallization inhibitor in order to facilitate dissolution.

Preferably in the process according to any embodiment of the present invention, the solvent has a boiling point (at atmospheric pressure) in the range of about 40° C. to about 120° C., more preferably in the range of about 50° C. to about 100° C., particularly about 50° C. to about 85° C., and most preferably about 50° C. to about 80° C. or about 55° C. to about 80° C. Preferably, the first or second solvent is mixed with the crystallization-inhibiting compound to form a solution or a dispersion (preferably a solution) of the compound in the solvent. When the crystallization-inhibiting compound is other than a polymer, it is preferably dissolved in the first or second solvent. The first and second solvents may be an organic solvent or an aqueous solvent comprising a surfactant. The amount of surfactant should be sufficient to dissolve the active ingredient in an aqueous solvent.

Preferably the at least one first and second solvent are an organic solvent. Preferably, the solvents have a low boiling point (i.e. 130° C. or less at atmospheric pressure). Preferably, the solvents have a boiling point of 120° C. or less, more preferably 100° C. or less, more preferably 90° C. or less, and particularly 80° C. or less at atmospheric pressure. Preferably, the solvents have a minimum boiling point of 40° C., more preferably 50° C., most preferably about 50° C. to about 80° C. Suitable solvents include but are not limited to C₁₋₃ alcohols, such as ethyl alcohol, and methyl alcohol, and C₃₋₄ ketones, such as acetone, or mixtures thereof. Particularly preferred solvents are ethanol and acetone.

As mentioned above, dissolution of the crystallization inhibiting compound can be facilitated by the addition of a surfactant to the mixture of the crystallization inhibiting compound in the second solvent. Suitable surfactants include but are not limited to: non ionic surfactants, such as: Polyoxyethylene Sorbitan Fatty Acid Esters, fatty acid esters of alcohols and copolymers of ethylene oxide and propylene oxide, anionic surfactants, such as: Sodium Lauryl Sulfate and Docusate Sodium and amphoteric agents such as phospholipids and polypeptides. Sodium lauryl sulfate is a preferred surfactant.

In particular, the surfactant may be useful in facilitating the dissolution of certain crystallization inhibitors such as gums, oils, waxes, fatty acids, and fatty acid esters. The surfactant may be used in small quantities, i.e. sufficient amounts to enable the dissolution of the crystallization inhibitor in the solvent. For example, suitable weight ratios of surfactant:crystallisation inhibitor are in the range of about 1:5 to 1:40, about 1:10 to 1:30, about 1:10 to 1:25 and preferably about 1:15 to 1:20.

In any embodiment of the present invention, the ratio between the gabapentin enacarbil and the at least one first solvent may be between about 0.01 g/ml and about 1 g/ml, more preferably about 0.03 g/ml and about 0.5 g/ml, more preferably about 0.03 g/ml to about 0.4 ml, or about 0.05 g/ml to about 0.3 ml, more preferably between about 0.05 g/ml and about 0.2 g/ml, more preferably about 0.06 g/ml and about 0.1 g/ml.

In any embodiment of the present invention the ratio between the at least one crystallization-inhibiting compound and the at least one second solvent may be between about 0.001 g/ml and about 2 g/ml, more preferably about 0.004 g/ml and about 1.5 g/ml, more preferably between about 0.006 g/ml and about 1.2 g/ml, more preferably about 0.008 g/ml and about 1.1 g/ml, more preferably between about 0.01 g/ml and about 0.8 g/ml and more preferably between about 0.3 g/ml and about 0.8 g/ml.

The stabilized composition of non-crystalline gabapentin enacarbil and at least one crystalline-inhibiting compound as defined in any of the embodiments described herein, can be used to prepared pharmaceutical dosage forms. The dosage forms can be prepared by providing the stabilized composition as defined in any of the embodiments discussed herein, and blending, or granulating the composition with at least one pharmaceutically acceptable excipient (preferably at least one carrier such as microcrystalline cellulose and/or ethylcellulose to form a granulate. Optionally, the granulate can be milled, and/or or the granulate can optionally be mixed with one or more further excipients including pharmaceutically acceptable excipients such as fillers, lubricants, glidants, binders and controlled release excipients (such as controlled-release polymers) before compressing the granulates into tablets.

Alternatively, in a further aspect of the present invention, the stabilized composition of non-crystalline gabapentin enacarbil and at least one crystallization-inhibiting compound can be provided directly onto a pharmaceutically acceptable carrier material, e.g. by a wet granulation process. Thus, the solutions of the gabapentin enacarbil and the crystalline inhibiting compounds as described above (preferably wherein the first and second solvents are the same, and both are ethanol) can be prepared and the solutions sprayed onto the carrier material (for example microcrystalline cellulose, such as Avicel® PH105), e.g. in a fluidized bed granulator, whilst the solvent is evaporated. Typically, the temperature in the fluid bed granulator is about 20° C. to about 80° C., preferably about 25° C. to about 50° C., and more preferably about 30° C. to about 45° C. The fluidized bed granulator may be operated at below atmospheric pressure, e.g. under a vacuum. The resulting granulate can optionally be milled, and/or mixed with further pharmaceutically acceptable excipients (for example a lubricant, filler, binder, controlled release excipients, and preferably a lubricant, particularly silicon dioxide or magnesium stearate), before being formed into the dosage form, e.g. by compressing into tablets.

In another aspect, the invention provides a method for manufacturing a composition (such as a pharmaceutical dosage form) comprising non-crystalline gabapentin enacarbil and at least one crystallization-inhibiting compound by wet granulation. Typically the process may involve providing the stabilized composition of non-crystalline gabapentin enacarbil and at least one crystalline-inhibiting compound according to any of the embodiments described herein, and carrying out a wet granulation of this composition with other pharmaceutically acceptable excipients. The other pharmaceutically acceptable excipients are described above, and preferably include at least one carrier and with a lubricant.

The wet granulation may be performed using a high shear mixer. Optionally, during wet granulation the liquid (e.g. ethanol) is gradually introduced into the vessel. The wet granulation may be performed using a one pot system where a mixing/granulating/drying are incorporated in one vessel. The wet granulation may be performed using spray granulation including, but not limited to, a fluidized bed dryer.

In another embodiment, the present invention provides a method for manufacturing a composition comprising a non-crystalline gabapentin enacarbil and at least one crystallization-inhibiting compound by hot melt extrusion or hot melt granulation.

A preferred method of manufacture comprises melting the at least one crystallization-inhibiting compound; when fully melted, add the gabapentin enacarbil and heat until fully melted; cool or allow to cool the melt until solid. The melting for example in a temperature of about 60° C. or more, between about 60° C. and about 200° C. The cooling is exemplified by room temperature. For example, the cooling may be conducted using any suitable cooling means, such as an ice bath or other cooling equipment. Optionally, all methods described in the present invention include milling of the solid, wherein the milling is preferably performed below 10° C., preferably between about −10° C. to about 8° C., about −5° C. to about 5° C., or about 0° C. to about 4° C.

A further method of manufacture comprises melting the gabapentin enacarbil, and adding at the least one crystallization-inhibiting compound as described above to the melt. After stirring the mixture, the mixture may be cooled e.g. to room temperature or below (e.g. using an ice bath or other cooling equipment). The solidified material can be milled, wherein the milling is preferably performed below 10° C., preferably between about −10° C. to about 8° C., about −5° C. to about 5° C., or about 0° C. to about 4° C.

In each of the above described methods, the resulting composition comprising the non-crystalline gabapentin enacarbil and at least one crystallization-inhibiting compound, either as a solid or milled solid, can be incorporated into a pharmaceutical composition by optionally admixing or blending the composition with one or more pharmaceutically acceptable excipients (for example a lubricant, filler, binder, controlled-release excipients, and preferably a lubricant, particularly silicon dioxide or magnesium stearate), before being formed into the dosage form, e.g. by compressing into tablets.

In another aspect, the present invention provides a method for manufacturing such a composition comprising a non-crystalline gabapentin enacarbil and at least one crystallization-inhibiting compound. Thus, a stabilized composition comprising non-crystalline gabapentin enacarbil and at least one crystallization-inhibiting compound according to any aspect of the present invention may be prepared by a process comprising the steps of:
(1) providing a solution comprising gabapentin enacarbil and the at least one crystallization-inhibiting compound and at least one organic solvent;
(2) removing the organic solvent(s).

Optionally, a pharmaceutical composition comprising the stabilized composition can be prepared by contacting the solution in step (1) with a pharmaceutically acceptable carrier before removal of the organic solvent(s). Thus, the method can comprise dissolving gabapentin enacarbil in at least one first solvent, mixing with at least one crystallization-inhibiting compound to obtain a mixture, followed by contacting a pharmaceutical acceptable carrier(s) with the mixed solution, for example by adding the mixture onto a pharmaceutical acceptable carrier(s), e.g. microcrystalline cellulose, to achieve wet particles, e.g. granules and further drying the solvent(s). Optionally, the obtained particles may further be milled at low temperature (below 10° C.) with at least one pharmaceutical acceptable excipient to obtain a pharmaceutical composition.

Optionally, the solution of the gabapentin enacarbil and the at least crystallization inhibitor compound may be provided by dissolution of the crystallization inhibitor compound in an organic solvent and dissolving the gabapentin enacarbil in the solution.

In any embodiment of the present invention where a solvent is used in the production process, any temperature at which the solvent is sufficiently liquid to dissolve the gabapentin enacarbil and, optionally, the at least one crystallization-inhibiting compound and the gabapentin enacarbil and the at least one crystallization-inhibiting compound are miscible in each other is suitable for manufacturing the composition of the present invention.

Any pharmaceutically acceptable method and equipment is suitable for the production of the solutions and dispersions (preferably solutions) of the present invention.

In any embodiment of the present invention where a solvent is used in the production process, the solvent(s) may be removed from the solution by techniques known in the art including but not limited to: distillation, evaporation, vacuum drying, oven drying, tray drying, rotational drying, spray drying, freeze-drying, fluid bed drying, flash drying, spin flash drying and thin-film drying. Preferably, the solvents are removed by evaporation or flash-evaporation techniques (preferably under vacuum) such as fluid bed drying. Freeze drying can also be used.

In any embodiment of the present invention where a solvent is used in the production process, when evaporation process is used to remove the solvent, preferably the temperature of the evaporation process is in the range between about 5° C. and about 150° C., more preferably between about 20° C. and about 120° C., more preferably between about 40° C. and about 110° C. and more preferably between about 50° C. and about 100° C.

In a preferred embodiment the storage conditions of the composition according to all aspects of the present invention are under temperature of not more than 10° C., for example, −20° C., −4° C., 4° C. and 7° C.

In any embodiment of the present invention the gabapentin enacarbil starting material may be any known form of gabapentin enacarbil. For example, crystalline gabapentin enacarbil or amorphous gabapentin enacarbil.

According to still another embodiment, the majority of gabapentin enacarbil, e.g. at least about 60%, at least about 70%, at least about 80%, at least about 90%, at least about 95%, at least about 99% by weight of the composition doesn't crystallize during solvent removal so that the formed solid composition is free of gabapentin enacarbil crystals, as confirmed by X-ray diffraction (XRD) [data or spectrum]. Thus, preferably, following manufacture, the stabilized composition or the pharmaceutical dosage forms do not contain detectable amounts of crystalline gabapentin enacarbil.

Having thus described the invention with reference to particular preferred embodiments and illustrated with the examples below, those skilled the art can appreciate modifications to the invention as described and illustrated that do not depart from the spirit and scope of the invention as disclosed in the specification.

The Examples are set forth to aid in understanding the invention but are not intended to, and should not be construed to, limit its scope in any way. The examples do not include detailed descriptions of conventional methods. All references mentioned herein are incorporated in their entirety.

### EXAMPLES

### Example 1

| | Formu- | Formu- | Formu- | Formu- |
|---|---|---|---|---|
| | lation 1 | lation 2 | lation 3 | lation 4 |
| Ingredient | Quantity | | | |
| Part 1 | | | | |
| Ethylcellulose | 0.3 g | 0.2 g | 0.15 g | 0.05 g |
| (Ethocel 7cps) | | | | |
| Acetone | 5 ml | 5 ml | 5 ml | 5 ml |
| Part 2 | | | | |
| Gabapentin | 0.3 g | 0.3 g | 0.3 g | 0.3 g |
| enacarbil | | | | |
| Acetone | 5 ml | 5 ml | 5 ml | 5 ml |
| XRD | Non-crystalline | | Non-crystalline | Crystalline |
| characterization | GABA-E | | GABA-E | GABA-E |

1. Dissolve Ethocel 7cps in acetone
2. Dissolve Gabapentin Enacarbil in Acetone.
3. Mix part 1 and part 2 to homogeneous mixture in Petri dish.
4. Evaporate the solvent using hot plate at 50-100° C. (preferably to dryness)
5. Cool down using ice bath.

Formulations 1,3, and 4 were prepared and tested at XRD at regular scan speed.

Formulation 2 is a prophetic example.

### Example 2

| | Formu- | Formu- | Formu- | Formu- | Formu- |
|---|---|---|---|---|---|
| | lation 5 | lation 6 | lation 7 | lation 8 | lation 9 |
| Ingredient | Quantity | | | | |
| Part 1 | | | | | |
| Ethylcellulose | 0.5 g | 0.33 g | 0.5 g | 0.15 g | 0.05 g |
| (Ethocel 100 cps) | | | | | |
| Acetone | | | 5 ml | 5 ml | 5 ml |
| Ethyl alcohol | 5 ml | 5 ml | | | |
| Part 2 | | | | | |
| Gabapentin | 0.5 g | 0.5 g | 0.5 g | 0.3 g | 0.3 g |
| enacarbil | | | | | |
| Acetone | 5 ml | 5 ml | 5 ml | 5 ml | 5 ml |
| XRD | Non- | | | Non- | Crystalline |
| characterization | crystalline | | | crystalline | GABA-E |
| | GABA-E | | | GABA-E | |

1. Dissolve Ethocel 100cps in acetone or ethyl alcohol.
2. Dissolve Gabapentin Enacarbil in Acetone.
3. Mix part 1 and part 2 to homogeneous mixture in Petri dish.
4. Evaporate the solvent using hot plate at 50-100° C.
5. Cool down using ice bath.

Formulations 5, 8, and 9 were prepared and tested at XRD at regular scan speed.

Formulations 6 and 7 are prophetic examples.

### Example 3

| | Formu- | Formu- | Formu- | Formu- | Formu- |
|---|---|---|---|---|---|
| | lation | lation | lation | lation | lation |
| | 10 | 11 | 12 | 13 | 14 |
| Ingredient | Quantity, g | | | | |
| Part 1 | | | | | |
| Glyceryl | 0.2 g | 0.05 g | | | |
| monostearate | | | | | |
| 402 BP | | | | | |
| Glyceryl | | | 0.3 g | 0.2 | 0.05 g |
| monostearate | | | | | |
| 40-55 EP | | | | | |
| Ethyl alcohol | 5 ml | 5 ml | 5 ml | 5 ml | 5 ml |
| Part 2 | | | | | |
| Gabapentin | 0.3 g | 0.3 g | 0.3 g | 0.3 g | 0.3 g |
| enacarbil | | | | | |
| Acetone | 5 ml | 5 ml | 5 ml | 5 ml | 5 ml |
| XRD | | Crystalline | Non- | | Crystalline |
| characterization | | GABA-E | crystalline | | GABA-E |
| | | | GABA-E | | |

1. Dissolve Glyceryl monostearate in ethyl alcohol.
2. Dissolve Gabapentin Enacarbil in Acetone.
3. Mix part 1 and part 2 to homogeneous mixture in Petri dish.
4. Evaporate the solvent using hot plate at 50-100° C.
5. Cool down using ice bath.

Formulations 11, 12 and 14 were prepared and tested using XRD at regular scan speed.

Formulations 10 and 13 are prophetic examples.

### Example 4

| | Formu- | Formu- | Formu- | Formu- | Formu- |
|---|---|---|---|---|---|
| | lation | lation | lation | lation | lation |
| | 15 | 16 | 17 | 18 | 19 |
| Ingredient | Quantity, g | | | | |
| Part 1 | | | | | |
| Hydrogenated castor | 0.33 g | 0.5 g | 0.5 g | 0.2 g | 0.05 g |
| oil | | | | | |
| Sodium lauryl | 0.02 g | 0.02 g | | | |
| sulfate | | | | | |
| Acetone | | | 5 ml | | 5 ml |
| Ethyl alcohol | 5 ml | 5 ml | | 5 ml | |
| Part 2 | | | | | |
| Gabapentin | 0.5 g | 0.5 g | 0.5 g | 0.3 g | 0.3 g |
| enacarbil | | | | | |
| Acetone | 5 ml | 5 ml | 5 ml | 5 ml | 5 ml |
| XRD | | Non- | Non- | | |
| characterization | | crystalline | crystalline | | |
| | | GABA-E | GABA-E | | |

1. Dissolve hydrogenated castor oil and sodium lauryl sulfate in ethyl alcohol or Acetone.
2. Dissolve Gabapentin Enacarbil in Acetone.
3. Mix part 1 and part 2 to homogeneous mixture in Petri dish.
4. Evaporate the solvent using hot plate at 50-100° C.
5. Cool down using ice bath.

Formulations 16 and 17 were prepared and tested using XRD at regular scan speed.

Formulations 15, 18 and 19 are prophetic examples.

### Example 5

| | Formu- | Formu- | Formu- | Formu- | Formu- |
|---|---|---|---|---|---|
| | lation | lation | lation | lation | lation |
| | 20 | 21 | 22 | 23 | 24 |
| Ingredient | Quantity, g | | | | |
| Part 1 | | | | | |
| Hydroxy- | 0.33 g | 0.33 g | 0.5 g | 0.15 g | 0.05 g |
| propylcellulose | | | | | |
| (Klucel LF) | | | | | |
| Acetone | | 5 ml | 5 ml | 5 ml | 5 ml |
| Ethyl alcohol | 5 ml | | | | |
| Part 2 | | | | | |
| Gabapentin | 0.5 g | 0.5 g | 0.5 g | 0.3 g | 0.3 g |
| enacarbil | | | | | |
| Acetone | 5 ml | 5 ml | 5 ml | 5 ml | 5 ml |
| XRD | | | Non- | Non- | Crystalline |
| characterization | | | crystalline | crystalline | GABA-E |
| | | | GABA-E | GABA-E | |

1. Dissolve Klucel LF in ethyl alcohol or Acetone.
2. Dissolve Gabapentin Enacarbil in Acetone.
3. Mix part 1 and part 2 to homogeneous mixture in Petri dish.
4. Evaporate the solvent using hot plate at 50-100° C.
5. Cool down using ice bath.

Formulations 22, 23 and 24 were prepared and tested using XRD at regular scan speed.

Formulations 20 and 21 are prophetic examples.

### Example 6

| | Formu- | Formu- | Formu- | Formu- | Formu- |
|---|---|---|---|---|---|
| | lation | lation | lation | lation | lation |
| | 25 | 26 | 27 | 28 | 29 |
| Ingredient | Quantity, g | | | | |
| Part 1 | | | | | |
| Povidone K90 | 0.33 g | 0.33 g | 0.5 g | 0.15 g | 0.05 g |
| Acetone | | 5 ml | | | |
| Ethyl alcohol | 5 ml | | 5 ml | 5 ml | 5 ml |
| Part 2 | | | | | |
| Gabapentin | 0.5 g | 0.5 g | 0.5 g | 0.3 g | 0.3 g |
| enacarbil | | | | | |
| Acetone | 5 ml | 5 ml | 5 ml | 5 ml | 5 ml |
| XRD | | | Non- | Non- | Non- |
| characterization | | | crystalline | crystalline | crystalline |
| | | | GABA-E | GABA-E | GABA-E |

1. Dissolve Povidone K90 in ethyl alcohol or Acetone.
2. Dissolve Gabapentin Enacarbil in Acetone.
3. Mix part 1 and part 2 to homogeneous mixture in Petri dish.
4. Evaporate the solvent using hot plate at 50-100° C.
5. Cool down using ice bath.

Formulations 27, 28 and 29 were prepared and tested using XRD at regular scan speed.

Formulations 25 and 26 are prophetic examples.

### Example 7

| | Formu- | Formu- | Formu- | Formu- |
|---|---|---|---|---|
| | lation | lation | lation | lation |
| | 30 | 31 | 32 | 33 |
| Ingredient | Quantity, g | | | |
| Part 1 | | | | |
| Povidone K30 | 0.2 g | 0.15 g | 0.05 g | 0.3 g |
| Ethyl alcohol | 5 ml | 5 ml | 5 ml | 5 ml |
| Part 2 | | | | |
| Gabapentin enacarbil | 0.3 g | 0.3 g | 0.3 g | 0.3 g |
| Acetone | 5 ml | 5 ml | 5 ml | 5 ml |
| XRD characterization | | Non- | Non- | Non- |
| | | crystalline | crystalline | crystalline |
| | | GABA-E | GABA-E | GABA-E |

1. Dissolve Povidone K30 in ethyl alcohol.
2. Dissolve Gabapentin Enacarbil in Acetone.
3. Mix part 1 and part 2 to homogeneous mixture in Petri dish.
4. Evaporate the solvent using hot plate at 50-100° C.
5. Cool down using ice bath.

Formulations 31, 32 and 33 were prepared and tested using XRD at regular scan speed.

Formulation 30 is a prophetic example.

### Example 8

| | Formu- | Formu- | Formu- | Formu- |
|---|---|---|---|---|
| | lation | lation | lation | lation |
| | 34 | 35 | 36 | 37 |
| Ingredient | Quantity, g | | | |
| Part 1 | | | | |
| Copovidone | 0.2 | 0.15 g | 0.05 g | 0.3 g |
| (Plasdone S630) | | | | |
| Acetone | 5 ml | 5 ml | 5 ml | 5 ml |
| Part 2 | | | | |
| Gabapentin enacarbil | 0.3 g | 0.3 g | 0.3 g | 0.3 g |
| Acetone | 5 ml | 5 ml | 5 ml | 5 ml |
| XRD characterization | | Non- | Crystalline | Non- |
| | | crystalline | GABA-E | crystalline |
| | | GABA-E | | GABA-E |

1. Dissolve Copovidone in Acetone.
2. Dissolve Gabapentin Enacarbil in Acetone.
3. Mix part 1 and part 2 to homogeneous mixture in Petri dish.
4. Evaporate the solvent using hot plate at 50-100° C.
5. Cool down using ice bath.

Formulations 35, 36 and 37 were prepared and tested using XRD at regular scan speed. Formulation 34 is a prophetic example.

### Example 9

| | Formulation | Formulation | Formulation |
|---|---|---|---|
| | 38 | 39 | 40 |
| Ingredient | Quantity | | |
| Povidone K90 | 0.05 g | | |
| Ethocel 7cps | | 0.05 g | |
| HPMC (Pharmacoat | | | 0.2 g |
| 606) | | | |
| Gabapentin enacarbil | 0.3 g | 0.3 g | 0.2 g |
| XRD characterization | Non- | Non- | Non- |
| | crystalline | crystalline | crystalline |
| | GABA-E | GABA-E | GABA-E |

1. Melt the polymer in a Petri dish on hot plate until full melting.
2. Add Gabapentin Enacarbil to the Petri dish and heat while mixing until melting.
3. Cool down using ice bath.
4. Mill using mortar and pestle.

Formulations 38, 39 and 40 were prepared and tested using XRD at regular scan speed.

### Example 10

Control-release tablet 300 mg

| Ingredient | Quantity, mg/tablet |
|---|---|
| Dry mixture from formulation 3 | 450.0 |
| Klucel MF | 120.0 |
| Avicel PH 102 | 65.0 |
| Magnesium Stearate | 3.0 |
| End Tablet weight | 638.0 |

1. Mix dried drug mixture from formulation 3, Klucel MF and Avicel PH 102 in a mixer for 15 minutes.
2. Screen Magnesium Stearate through a suitable screen, add to the blend and mix for 5 minutes.

Press the final blend to the tablets using suitable tools.

Example 10 is a prophetic example.

### Example 11

A composition containing Gabapentin Enacarbil, PVP K90 (crystallization-inhibiting compound) and Avicel PH105 (carrier).

Dissolve the PVP K90 (10 g) in about 100 ml Ethyl Alcohol while stirring for about 30 min. Add Gabapentin Enacarbil (60 g) into the solution and stir until fully dissolve.

Separately, put Avicel PH105 (40 g) into a fluidized air bed granulator and heat up to a temperature of 40° C.

Spray the Gabapentin Enacarbil solution onto the Avicel PH 105 while the ethyl alcohol is evaporated.

The granulate thus obtained can be milled, mixed with additional excipients and transformed into tablets. Any suitable conventional technique for preparing such dosage forms can be used.

Example 11 is a prophetic example.

### Example 12

A composition containing GABA-E, mixture of PVP K90, PVP K30 and PVP K25 ((crystallization-inhibiting compounds) and Avicel PH105 ((carrier).

Dissolve PVP K90 (3 g), PVP K30 (4 g) and PVP K25 (3 g) in Ethyl Alcohol (about 100 ml) while stirring for about 30 min. Add GABA-E (60 g) into the solution and stir until fully dissolve.

Separately, put Avicel PH105 (40 g) into a single pot granulator and heat up to a temperature of 40° C.

Gradually introduce the GABA-E onto the Avicel PH 105, while drying (optionally under vacuum) each of the added portion before addition of the next portion.

The granulate thus obtained can be milled with aerosil at low temperature [i.e. (−10) to (−20° C.)], then optionally mixed with additional excipients and compressed into tablets. Any suitable conventional technique for preparing such dosage forms can be used.

Example 12 is a prophetic example.

### Example 13

2.16 g of PVP K90 and 2.16 g of PVP K30 were dissolved in about 20 mL ethyl alcohol, during stirring and heating to about 60° C. using hot plate.

9.0 g of PVP VA64 were dissolved in about 20 mL of Acetone during stirring and heating to about 60° C. using hot plate.

3.6 g of Gabapentin Enacarbil were dissolved in about 15 ml Acetone.

All the three mentioned solutions were mixed together and part of the solvents was evaporated (25 mL of solution was left).

The mixed solution was slowly poured on 3.6 g of microcrystalline cellulose during mixing using mortar and pestle and dried using vacuum oven at 50° C. 3.0 g of aerosil (silicon dioxide) was added to the dry mixture and the dry mixture was kept over night at −20° C.

The freeze mixture was milled using mortar and pestle under N2 until uniform blend was obtained.

Tablets containing 300 mg of Gabapentin enacarbil were prepared using tablet machine (single punch).

The tablets were kept at room temperature (25° C.) and at 30° C. and 75% relative humidity for 1 week and the XRD of the tablets was tested (according to the method below).

No peaks of gabapentin enacarbil were observed at both samples.

### Example 14

12 g of Copovidone (PVP VA64) was dissolved in 250 mL of Acetone and 30 g of Gabapentin enacarbil was add to the solution and mixed until the Gabapentin enacarbil was fully dissolved.

The solution was slowly poured on 30 g of microcrystalline cellulose (Avicel PH113) during mixing using mortar and pestle. The mixture (sticky suspension) was dried using vacuum oven at 50° C. During the drying process the mixture was manually mixed several times in order to prevent lumps.

2.5 g of silicon dioxide (Aerosil) was added to the dried mixture and the mixture was frozen to −20° C. over night.

The frozen mixture was milled using small milling machine. Tablets containing 300 mg of Gabapentin enacarbil were prepared using tablet machine (single punch).

No peaks of gabapentin enacarbil were observed in XRD measurements at zero time and after storage at the following conditions and time intervals:

(1) 25° C./60% relative humidity at 3 and 6 months.

(2) 30° C./65% relative humidity at 3 and 6 months.

(3) 40° C./75% relative humidity at 1, 2, 3 and 6 months.

### Example 15

3.96 g of Povidone K30 and 3,96 g of Povidone K90 were dissolved in 300 mL ethyl alcohol, 11 g of Copovidone (PVP VA64) was dissolved in 160 mL of Acetone. Both solutions were mixed together and 66 g of gabapentin enacarbil was add to the solution mixture and mixed until the Gabapentin enacarbil was fully dissolved.

The solution was slowly poured on 66 g of microcrystalline cellulose (Avicel PH113) during mixing using mortar and pestle, The mixture was dried using vaccum oven at 50° C. During the drying process the mixture was manually mixed several times in order to prevent lumps.

5.5 g of silicone dioxide (Aerosil) was added to the dried mixture and the mixture was frozen to −20° C. over night.

The frozen mixture was milled using small milling machine,

8.017 g of Hypromellose having a viscosity of 80,000-120,000 meas (Methocel K100M) was added to 57 g of the dry milled mixture.

Tablets of 811 mg (tablet weight) containing 300 mg of Gabapentin enacarbil were prepared using tablet machine (single punch).

No peaks of gabapentin enacarbil were observed in XRD measurements at zero time and after storage at the following conditions and time intervals:

(4) 25° C./60% relative humidity at 3 months

(5) 30° C./65% relative humidity at 3 and 6 months.

(6) 40° C./75% relative humidity at 1, 2, 3 and 6 months.

XRD Sethod

X-Ray Powder Diffraction Analysis (XRPD) was measured with a Philips X'Pert PRO powder diffractometer using the following parameter.

| | |
|---|---|
| Sample holder | Sample is applied directly on silicon |
| preparation | PW1817/32 “zero background” holder |
| Instrument | Philips X′Pert PRO |
| X-Ray tube | PW3373/00; Cu anode LFF |
| X-ray radiation | λ (CuKα₁) = 1.540598 Å |
| Temperature | 295 ± 5 K |
| Step size (regular scan) | 0.00167 |
| Scan speed (regular | 0.05°/sec |
| scan) | |
| Step size (slow scan) | 0.00167 |
| Scan speed (slow scan) | 0.002°/sec |

1. A stabilized composition comprising a non-crystalline gabapentin enacarbil and at least one crystallization-inhibiting compound.

2. The composition of claim 1, wherein the weight ratio of the non-crystalline gabapentin enacarbil to the at least one crystallization-inhibiting compound is between about 1:1 and about 10:1.

3. The composition of claim 1 or claim 2, wherein the weight ratio of the non-crystalline gabapentin enacarbil to the at least one crystallization-inhibiting compound is at least about 1.

4. The composition of claim 1, wherein the composition comprises between about 50% to about 100% by weight of the non-crystalline gabapentin enacarbil, based on the total amount of the non-crystalline gabapentin enacarbil and the at least one crystallization-inhibiting compound.

5. The composition of claim 1, wherein the non-crystalline gabapentin enacarbil and the at least one crystallization-inhibiting compound are in an intimate admixture, or a solid solution.

6. A composition according to claim 1 wherein the crystallization-inhibiting compound is selected from a polymer, waxes, gums, oils, fatty acids, fatty acid esters, or a mixture thereof.

7. A composition according to claim 1 wherein the crystallization-inhibiting compound is a polymer selected from a pyrrolidone polymer, cross-linked polyvinylpyrrolidone or a mixture thereof.

8. A composition according to claim 1 wherein the crystallization-inhibiting compound is a cellulose ether, and preferably ethylcellulose, hydroxyethylcellulose, hydroxypropylcellulose, hydroxypropylmethyl cellulose, or a mixture thereof.

9. A composition according to claim 1 wherein the crystallization-inhibiting compound is a wax, beeswax, carnauba wax, microcrystalline wax, or a mixture thereof.

10. A composition according to claim 1 wherein the crystallization-inhibiting compound is a gum.

11. A composition according to claim 1 wherein the crystallization-inhibiting compound is an oil, preferably hydrogenated castor oil.

12. A composition according to claim 1 wherein the crystallization-inhibiting compound is a fatty acid.

13. A composition according to claim 1 wherein the crystallization-inhibiting compound is a fatty acid ester.

14. A composition according to claim 1 wherein the crystallization-inhibiting is selected from a glyceryl monostearate, hydrogenated castor oil, polyvinylpyrrolidone, cross-linked polyvinylpyrrolidone, ethylcellulose, hydroxypropyl cellulose, hydroxypropyl methylcellulose or a mixture thereof.

15. The composition of claim 1 comprising one, two or three crystallization-inhibiting compounds.

16. The composition of claim 1 wherein the composition consists essentially of non-crystalline gabapentin enacarbil and one or more crystallization-inhibiting compound(s).

17. The composition of claim 1 wherein the composition further comprises at least one pharmaceutically acceptable excipient.

18. A composition according to claim 17 wherein the surfactant is selected from non ionic surfactants, anionic surfactants, and amphoteric surfactants.

19. A composition according to claim 17 wherein the surfactant is sodium lauryl sulfate.

20. A process for preparing a stabilized composition comprising a non-crystalline gabapentin enacarbil and at least one crystallization-inhibiting compound, said process comprising:
(1) providing a solution comprising gabapentin enacarbil and the at least one crystallization-inhibiting compound in at least one organic solvent; and
(2) removing the at least one solvent.

21. A process according to claim 20 comprising:
(a) dissolving the gabapentin enacarbil in at least one first solvent,
(b) dissolving or mixing, preferably dissolving, at least one crystallization-inhibiting compound in at least one second solvent,
(c) mixing the two mixtures prepared in steps (a) and (b), and
(d) removing the solvents.

22. A process according to claim 21 wherein a surfactant is added to the mixture of the at least one crystallization-inhibiting compound in the at least one second solvent.

23. A process according to claim 21 wherein the first solvent and second solvent are the same or different, and each has a boiling point of about 40° C. to about 120° C.

24. A process according to claim 20 wherein the solvents are selected from C₁₋₃ alcohols, acetone or mixtures thereof.

25. The process of claim 20, wherein the solvents are removed by distillation, evaporation, vacuum drying, oven drying, tray drying, rotational drying, spray drying, freeze-drying, fluid bed drying, flash drying, spin flash drying, agitated vacuum drying or thin-film drying.

26. A process according to claim 20 wherein the solvents are removed at a temperature of about 5° C. to about 130° C.

27. A process for preparing a stabilized composition comprising a non-crystalline gabapentin enacarbil and at least one crystallization-inhibiting compound, said process comprising:
(A) providing a melted mixture of at least one crystallization-inhibiting compound and gabapentin enacarbil; and
(B) solidifying the melted mixture by cooling.

28. A process according to claim 27 comprising:
melting the at least one crystallization-inhibiting compound,
when fully melted, adding the gabapentin enacarbil and heating until fully melted, and
cooling the melt until it has solidified.

29. A process according to claim 27 comprising:
melting the gabapentin enacarbil;
adding the at least one crystallization-inhibitor compound, and
cooling the melted mixture until it has solidified.

30. A process according to claim 20 further comprising milling the composition.

31. A process according to claim 30 wherein the milling is carried out at a temperature below 10° C.

32. A pharmaceutical formulation comprising the composition of claim 1.

33. A pharmaceutical formulation according to claim 32 further comprising at least one pharmaceutically acceptable excipient.

34. The formulation of claim 33 wherein the at least one pharmaceutically acceptable excipient comprises at least one carrier and/or at least one lubricant.

35. The formulation of claim 31 wherein the pharmaceutically acceptable excipient includes at least one release retarding ingredient.

36. A process for preparing a pharmaceutical composition of 35 comprising:
(1) providing a stabilized composition comprising a non-crystalline gabapentin enacarbil and at least one crystallization-inhibiting compound, and
(2) admixing the composition with the at least one pharmaceutically acceptable excipient.

37. A process according to claim 36, wherein step (2) comprises mixing, blending, dry granulating, wet granulating or milling the stabilized composition with the at least one pharmaceutically acceptable excipient.

38. A process for preparing a pharmaceutical composition comprising:
(1) providing a solution comprising gabapentin enacarbil and the at least one crystallization-inhibiting compound in at least one organic solvent;
(2) contacting the solution of step (1) with at least one pharmaceutically acceptable excipient (preferably a carrier); and
(3) removing the solvent(s).

39. A process according to claim 38 comprising:
dissolving the gabapentin enacarbil in at least one first solvent,
dissolving or mixing, preferably dissolving, at least one crystallization-inhibiting compound in at least one second solvent optionally containing a surfactant,
mixing the two mixtures, to obtain a mixed solution,
contacting a pharmaceutical acceptable carrier(s) with the mixed solution, and
removing the solvents.

40. A process according to claim 38 wherein the solvent(s) are selected from C₁₋₃ alcohols, acetone or mixtures thereof, or each of the solvent(s) has a boiling point of about 40° C. to about 120° C.

41. A process according to claim 38 wherein the solvent is removed by fluid bed drying, evaporation, or tray drying, and preferably by fluid bed drying.

42. The process of claim 36, further comprising milling the produced composition.

43. The process of claim 36 further comprising blending the composition with one or more pharmaceutically acceptable excipients, preferably a lubricant, a diluent, a filler, a binder, a glident, a disintegrant, or a controlled release compound.

44. The process according to claim 36, further comprising compressing the composition to form a tablet.

45. A composition obtainable by a process according to claim 20 or 36.

46. A composition according to claim 1, wherein the composition contains less than about 1% by weight of crystalline gabapentin enacarbil:
at manufacture (time zero), or after 3 months of storage at 25° C. and 60% relative humidity (RH).

47. A composition according to claim 46 wherein the composition contains less than about 1% by weight of crystalline gabapentin enacarbil:
at manufacture (time zero), or after 3 months of storage at 40° C. and 75% relative humidity (RH).

48. A composition according to claim 47 wherein the composition contains less than about 1% by weight of crystalline gabapentin enacarbil:
at manufacture (time zero), or after 6 months of storage at 40° C. and 75% relative humidity (RH).

49. A dosage unit comprising the composition of claim 1.

50. (canceled)

51. A dosage unit comprising the formulation of claim 32.
